(19) United States
(12) Patent Application Publication
(10) Pub. No.: US 2020/0253787 A1
(43) Pub. Date: Aug. 13, 2020

(54) Bandage Having A Monitoring Device

(71) Applicant: BandGrip, Inc., Chicago, IL (US)

(72) Inventors: Keith Hoglund, Burr Ridge, IL (US); Tom Pruter, Lincolnshire, IL (US); Fred Smith, Houston, TX (US)

(21) Appl. No.: 16/746,651

(22) Filed: Jan. 17, 2020

Related U.S. Application Data
(60) Provisional application No. 62/793,526, filed on Jan. 17, 2019.

(51) Int. Cl. A61F 13/02; A61B 5/01; A61B 5/00

(52) U.S. Cl. CPC A61F 13/0233; A61B 5/6839; A61B 5/01

(57) ABSTRACT

A wound closure device comprising a mechanical coupling system and a system for monitoring the wound. The mechanical coupling system comprises a plurality of gripping structures; and a system for monitoring a wound including a communication device and a sensor.

### CROSS-REFERENCE TO RELATED APPLICATION

This application claims priority from U.S. Pat. App. Ser. No. 62/793,526 filed Jan. 17, 2019, entitled “BANDAGE HAVING A MONITORING DEVICE”, the entire specification of which is hereby incorporated by reference in its entirety.

This application is related to U.S. patent application Ser. No. 15/349,484, filed Nov. 11, 2016, entitled “Bandage”. This application is related to U.S. patent application Ser. No. 15/801,529, filed Nov. 2, 2017, entitled “Bandage and Anchor for Bandages”. This application is related to U.S. patent application Ser. No. 16/191,055, filed Nov. 14, 2018, entitled “Mechanical Tissue Coupling System, Applicator Therefor and Method for Use Thereof”. The entire specification of each of the foregoing is hereby incorporated by reference in its entirety.

### BACKGROUND OF THE DISCLOSURE

### 1. Field of the Disclosure

The disclosure relates in general to the treatment of wounds and the sensing of conditions at or near the wound, as well as communication of the conditions to an outside device, such as a smartphone, a tablet and/or a computer. Such devices may be topically applied (to wounds on the skin of the user), and the principles can also be directed to subcutaneous wounds.

### 2. Background Art

The use of bandages is well known in the art. Generally, bandages include a covering material and a base substrate. The covering material is coupled to the base substrate and the base substrate is adhered to the skin of a user.

Often, a wound that is deep may require stitches or the like to provide the necessary closure. Problematically, a typical bandage while helpful, often does not provide any, or sufficient force on a wound to direct the opposing sides of the wound to meet or to otherwise foster closure. Some bandages have been developed that include structures that engage the epidermis and the dermis so as to anchor portions of the bandage thereto.

While such bandages have been developed, many of such bandages lack sufficient retaining strength to be anchors, or, are excessively intrusive and can, themselves lead to injury and infection.

Additionally, it is difficult to have the information and data pertaining to various parameters around the wound. Current solutions generally either sense conditions that are too remote from the wound, or, lack the sensing capability to provide remote monitoring of the wound, or to monitor changes to the condition of the wound.

### SUMMARY OF THE DISCLOSURE

A wound closure device comprising a mechanical coupling system and a system for monitoring the wound. The mechanical coupling system comprises a plurality of gripping structures; and a system for monitoring a wound including a communication device and a sensor.

In some configurations, the communication device may comprise a display member, such as a color change, a light change or activation, a chemical change or a change in audible, visual and/or sensory configuration.

In some configurations, the transmitting device may comprise circuitry that can transmit/receive various different communication protocols, including, but not limited to Bluetooth, wife, zigbee, RF, among others.

The sensor may comprise a temperature sensor, a water sensor, a chemical sensor, among others.

### BRIEF DESCRIPTION OF THE DRAWINGS

The disclosure will now be described with reference to the drawings wherein:

FIG. 1 of the drawings is a bottom plane view of the bandage;

FIGS. 1a through 1e disclosed other configurations of the base substrate, the wound covering member and the mechanical and adhesive coupling systems;

FIG. 2 of the drawings is a perspective view of the mechanical coupling system;

FIG. 3 of the drawings is a perspective view of the mechanical coupling system;

FIG. 4 of the drawings is a side elevation view of the mechanical coupling system;

FIG. 5 of the drawings is a back elevational view of the mechanical coupling system;

FIG. 6 of the drawings is a front elevational view of the mechanical coupling system;

FIG. 7 of the drawings is a top plane view of the mechanical coupling system showing, in particular, an upstanding structure;

FIG. 8 of the drawings is a cross sectional view of the gripping structure, taken generally about lines 8-8 of FIG. 4, showing, in particular, the base configuration thereof;

FIG. 9 of the drawings is a partial perspective view of the gripping structure showing, in particular, the upstanding structure;

FIG. 10 of the drawings is a perspective view of the gripping structure showing, in particular, an upstanding structure;

FIG. 11 of the drawings is a perspective view of the gripping structure showing, in particular, an upstanding structure;

FIG. 12 of the drawings is a cross sectional view of the gripping structure taken generally about lines 12-12 of FIG. 7, showing, in particular, an upstanding structure and the tip;

FIG. 13 of the drawings is a perspective cross sectional view of the gripping structure taken generally about lines 13-13 of FIG. 7, showing, in particular, the overhanging portion;

FIG. 14 of the drawings is a perspective cross sectional view of the gripping structure taken generally about lines 14-14 of FIG. 7, showing, in particular, the overhanging portion;

FIG. 15 of the drawings is an exploded perspective view of another configuration of the bandage of the present disclosure;

FIG. 16 of the drawings is bottom plan view of the configuration of the bandage shown in FIG. 15.

### DETAILED DESCRIPTION OF THE DISCLOSURE

While this disclosure is susceptible of embodiment in many different forms, there is shown in the drawings and described herein in detail a specific embodiment(s) with the understanding that the present disclosure is to be considered as an exemplification and is not intended to be limited to the embodiment(s) illustrated.

It will be understood that like or analogous elements and/or components, referred to herein, may be identified throughout the drawings by like reference characters. In addition, it will be understood that the drawings are merely schematic representations of the invention, and some of the components may have been distorted from actual scale for purposes of pictorial clarity.

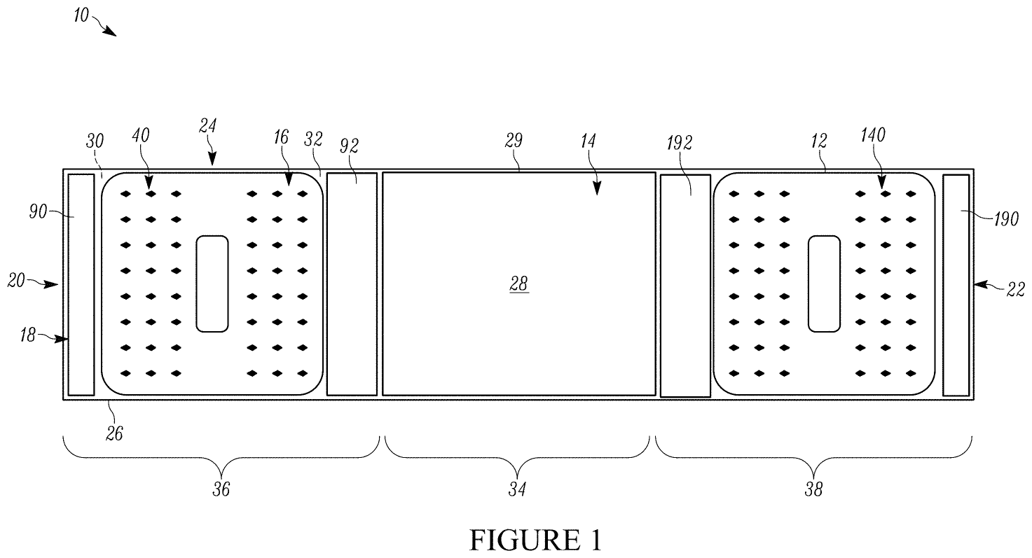

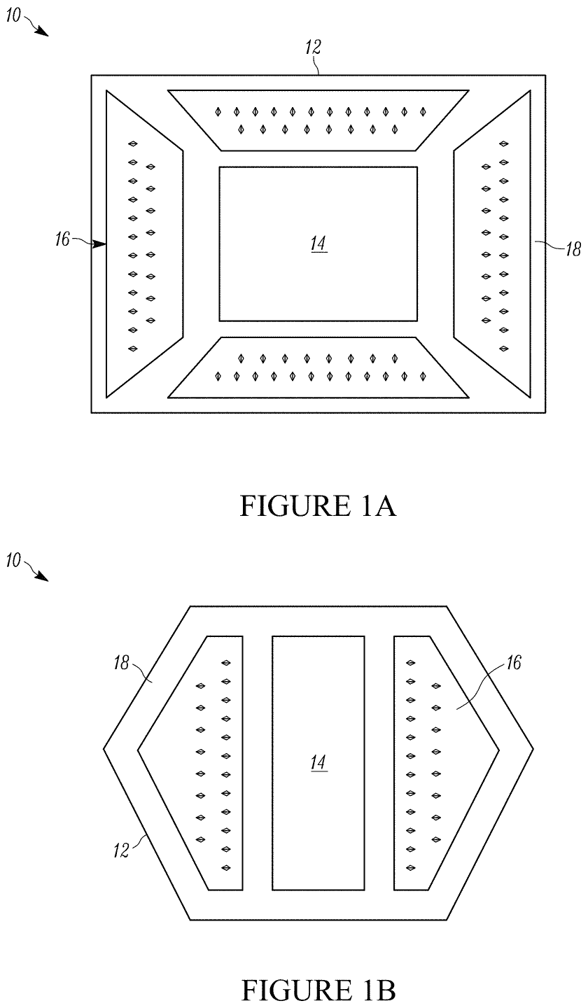

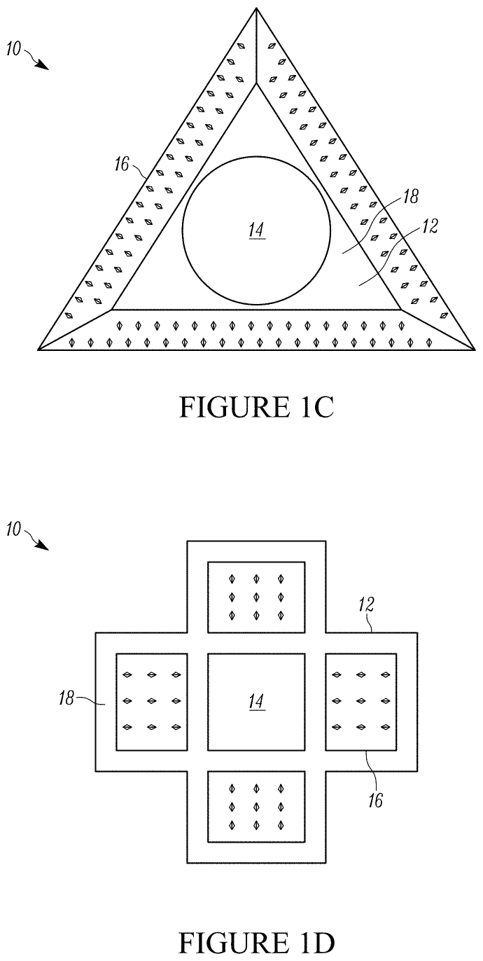

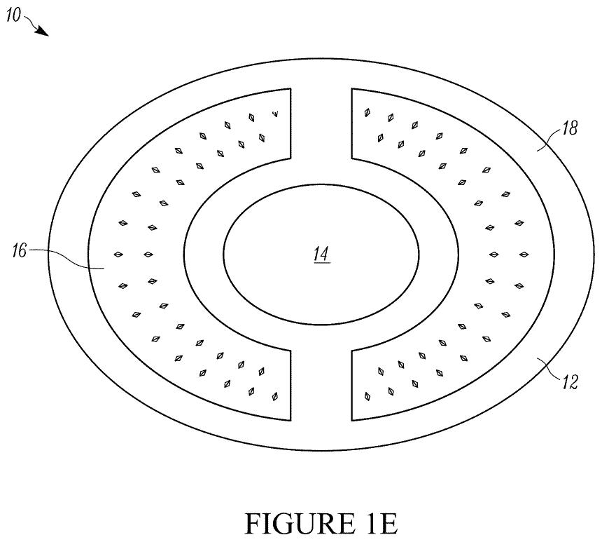

Referring now to the drawings and in particular to FIG. 1, the bandage is shown generally at 10. The bandage 10 comprises base substrates 12, wound covering member 14, mechanical coupling member 16, and adhesive coupling system 18. The base substrate 12 is sized in such a way that allows for the wound covering member 14, mechanical coupling system 16, and, optionally, adhesive coupling system 18 to be relatively positioned and maintained relative to each other.

Base substrate 12, shown in FIG. 1, comprises a first end 20, second end 22, first side edge 24, second side edge 26, outer surface 30, inner surface 32, central region 34, first side region 36, and second side region 38. First end 20 and second end 22 are generally opposite and parallel with reference to one another. The first side edge 24, second side edge 26, outer surface 30, and inner surface 32 extend substantially from first end 20 to second end 22, with the first side edge 24 and second side edge 26 generally opposite and parallel with reference to one another. Further, the outer surface 30 and inner surface 32 are generally opposite with reference to one another, with the wound covering member, the mechanical coupling system and the adhesive coupling system extending from the inner surface 32.

The regions of the bandage 10 are divided into three sections, known as the central region 34, first side region 36, and second side region 38. The central region 34 is substantially between the first side region 36 and second side region 38. In the configuration shown, the base substrate comprises a rectangular member. It will be understood that the base substrate may comprise additional different configurations, including, but not limited to square, triangular and other configurations. Some exemplary configurations of the base substrate (with suitably oriented wound covering members, mechanical coupling systems and adhesive coupling systems) are shown in FIGS. 1a through 1e. These are intended solely to be exemplary, and the disclosure is not limited to the configurations shown.

The base substrate 12 may be made of any suitably elastic material, such that elastic deformation is allowable to an extent that allows substantial alteration of the initial dimensions of the substrate 12. Elastic qualities in materials are known to be materials that can deform a given dimension, such as the length, but still possessing the ability to return to its initial dimension without harm to the structure of the material. In this case, an elastic material for base substrate 12 is offering allowance for the dimensions of the substrate, including but not limited to the length and width, to be altered and leaving room for the bandage 10 to attempt to return to its original dimensions. Such an elastic material can further provide an inward biasing force once installed on a patient so as to further aid in the closure of the wound (with the mechanical coupling, at least in part, resisting the biasing force). Materials contemplated include polymer films, natural or synthetic loose fibers that extend between the mechanical coupling members, as well as woven fabric materials, among others. The disclosure is not limited to any particular type of material from which the base substrate can be formed. In some configurations, the base substrate may have limited elasticity in one or more directions.

The wound covering member 14, shown in FIG. 1, comprises wound contact surface 28 and outer perimeter 29. The wound contact surface 28 faces generally outward from the inner surface 32 of the base substrate 12 and is surrounded by the outer perimeter 29. The outer perimeter 12 is substantially between the first side edge 24 and second side edge 26 of the base substrate, such that the wound covering member 14 is contained within the base substrate 12 (although it is contemplated that the wound covering member may extend beyond the perimeter of the base substrate. Further, the wound covering member 14 is generally within the central region 34 (while variations are contemplated). The wound covering member 14 may be composed of any suitable material that absorbs interstitial or other bodily fluids for which a bandage 10 may be used or which may supply other therapeutic benefits, such as medicament application, wound closure, among others. It is contemplated that a gauze, fiber or other material may be utilized for the wound covering member. In other configurations, a wound covering member may be omitted, with the base substrate overlying and in some cases abutting the wound. In still other configurations, an opening may be provided in the base substrate within the central region, with a wound covering member being applied separately over the base substrate and through the opening in the central region. Furthermore, it is contemplated that a plurality of wound covering members may be positioned over the central region, each being the same or having different properties and functions.

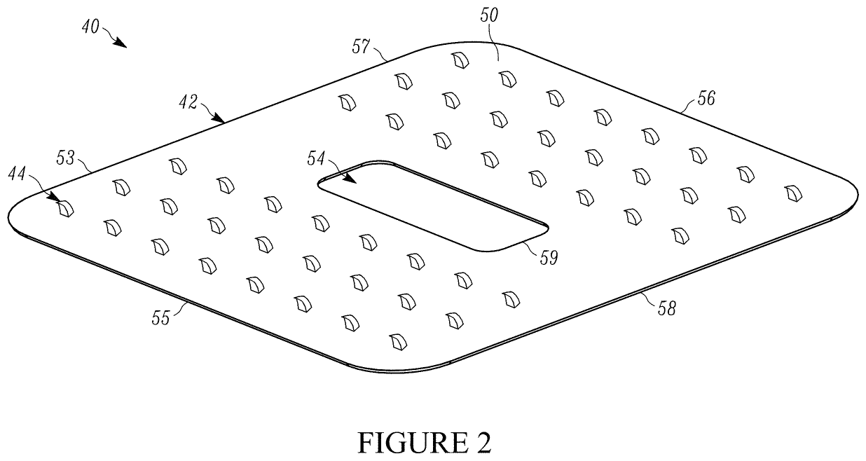

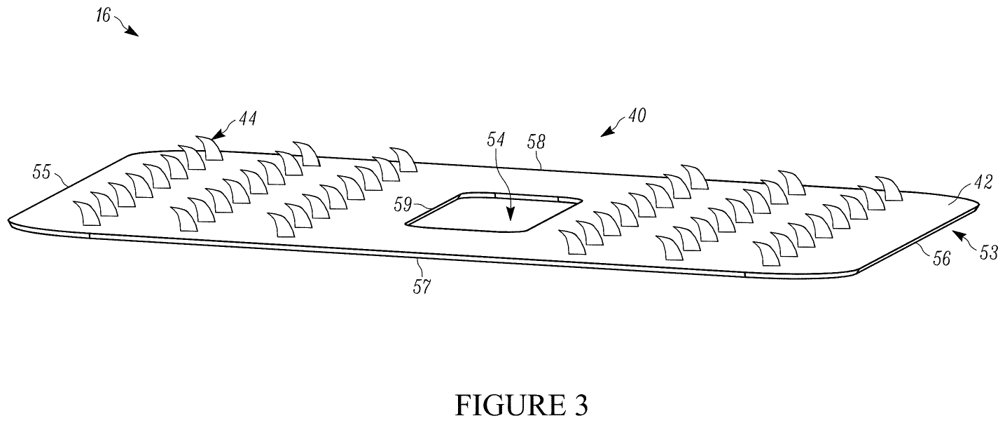

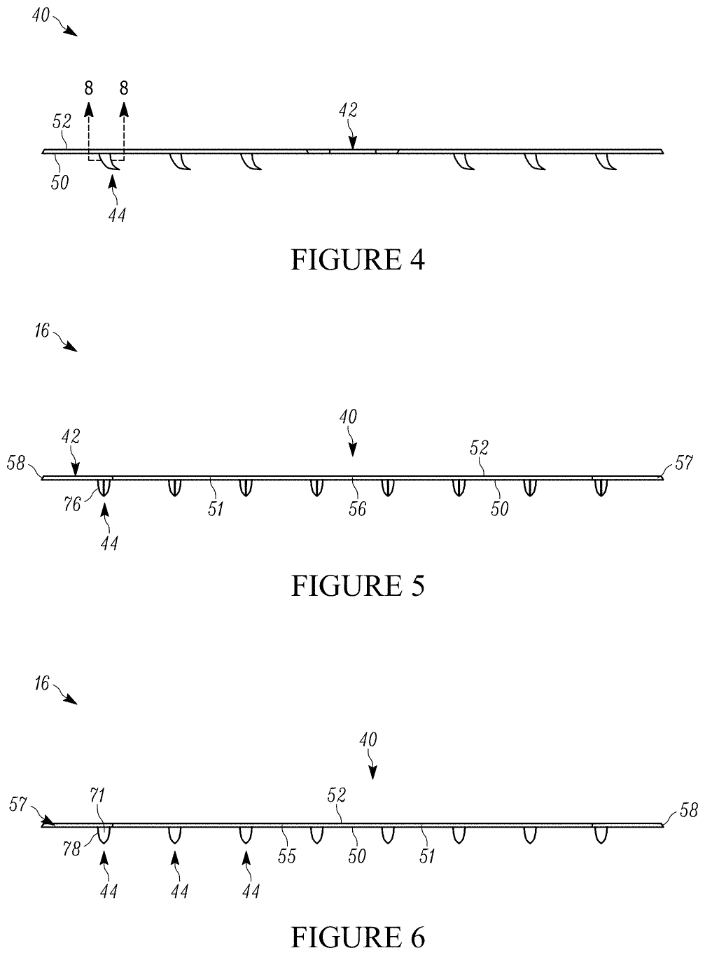

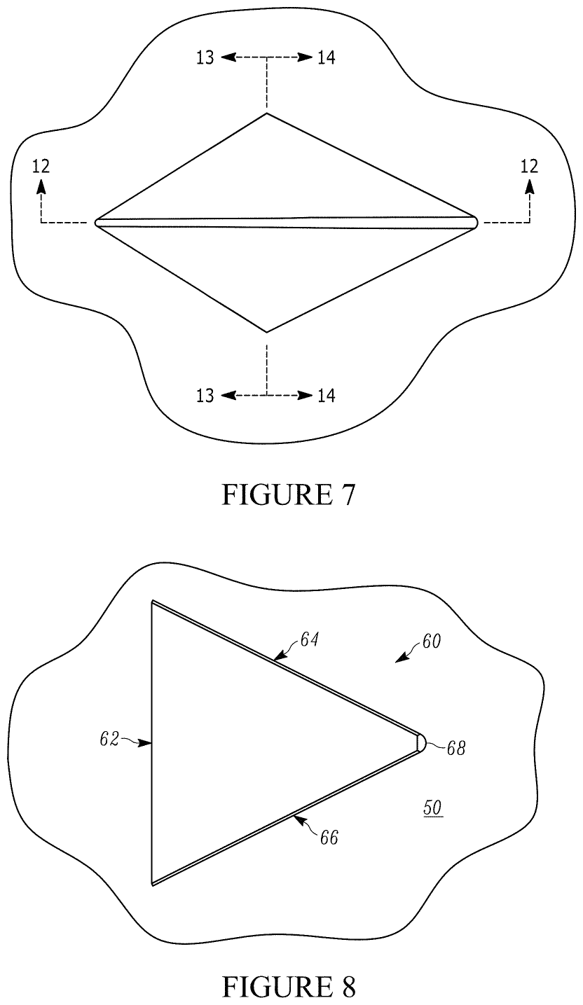

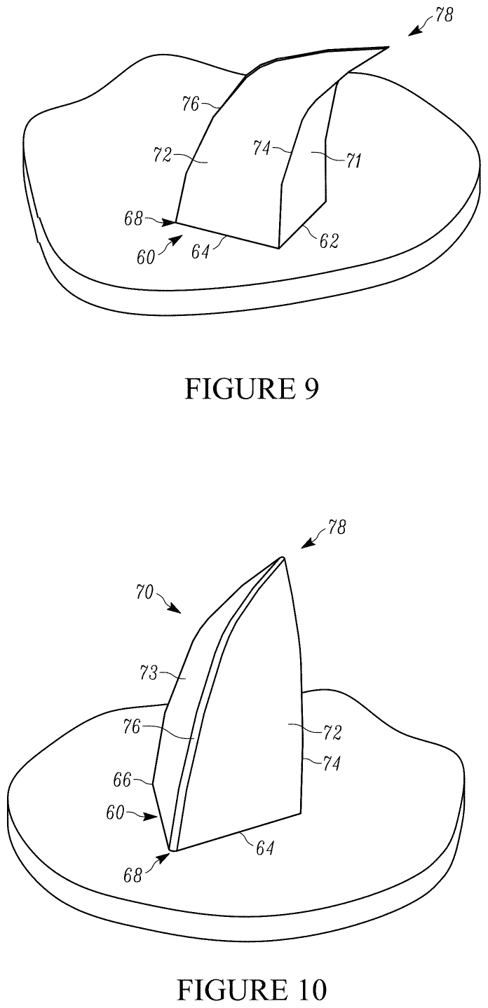

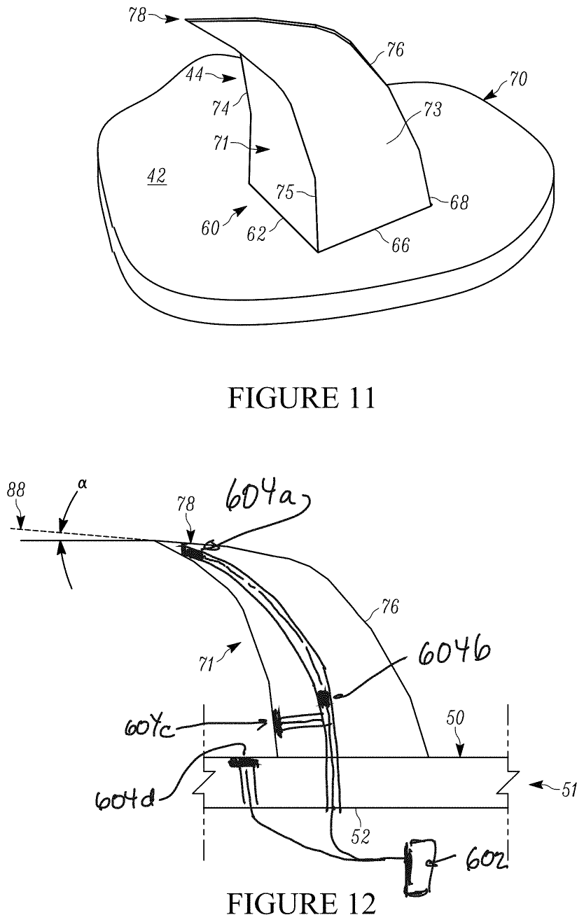

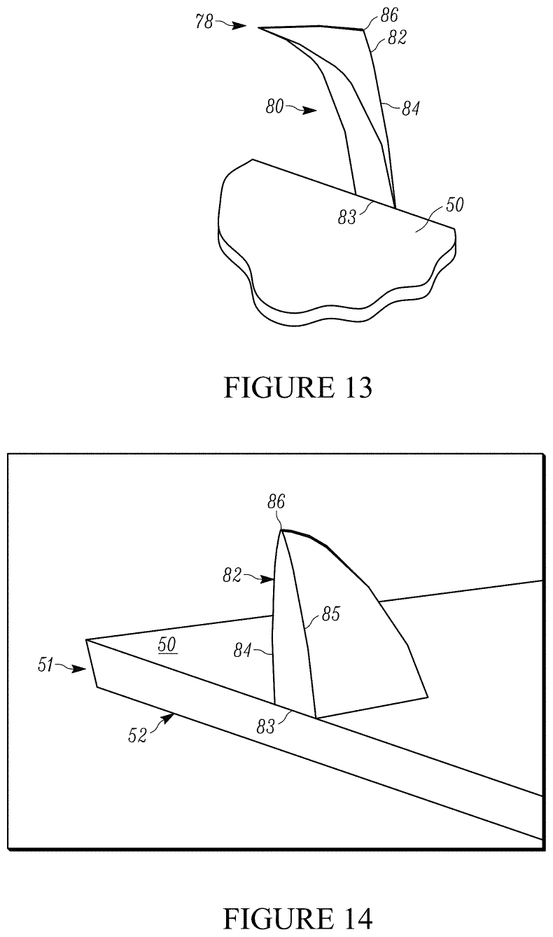

Shown in detail in FIGS. 2 through 13, the mechanical coupling system 16 comprises first side mechanical coupling member 40 and second side mechanical coupling member 140. It will be understood that in the configuration shown, the first side mechanical coupling member 40 and the second side mechanical coupling member are substantially identical mirror images of each other. Thus, in the present disclosure, the first side mechanical coupling member will be described with the understanding that the second side mechanical coupling member is a mirror image thereof, and, as such has substantially similar features and configurations. It will be understood that a bandage may have more than two mechanical coupling members, and that the configuration of each may be identical or different, with the understanding that generally, the mechanical coupling system has a base and gripping structures.

It will further be understood that the first side mechanical coupling member 40 and the second side mechanical coupling 140 may be bonded to the inner surface 32 of the base substrate in a number of different manners, including, but not limited to, adhesion, heat sealing, RF welding, co-molding or the like. In some configurations, the adhesive coupling system may comprise the same adhesive material with which the mechanical coupling members are attached to the base substrate.

The first side mechanical coupling member 40 comprises a base 42 and a plurality of gripping structures, such as gripping structure 44. As will be explained, the mechanical coupling member is integrally formed, through, injection molding of a polymer, such as Lexan®. In the configuration shown, the mechanical coupling system comprises a clear polymer, however, it is contemplated that translucent or opaque polymer constructions are likewise contemplated. The base 42 comprises a lower surface 50, upper surface 52, outer perimeter 54, and grasping opening 54. The outer perimeter is defined by leading edge 55, trailing edge 56, first side edge 57, and second side edge 58. The grasping opening 54 is defined in the base 42, and in particular, surrounded perimeter 59. It will be understood that the base substrate may include openings that generally correspond to the grasping opening. The lower surface 50 and upper surface 52 cooperatively define the thickness 51 of the base 42.

While not limited thereto, in the configuration shown, the base 42 comprises a square with rounded corners. The square has a nominal length and width of 0.875 inches. The grasping opening is generally centrally located and has a rectangular configuration with rounded inner corners, with nominal dimensions of 0.375 inches in width and 0.125 inches in depth. Both the outer perimeter and the perimeter of the grasping member are inclined at an angle of 30° such that the upper surface 52 is slightly smaller than the lower surface 50. In the configuration shown, the nominal thickness of the base is 0.005 inches with a range of 0.004 through 0.006 inches. It is contemplated that the dimensions may be varied of the base depending on the configuration of the overall bandage and the like. It will further be understood that the desire is to make the base have a thickness that allows for the flexibility necessary to follow the contours of the body.

The gripping structures are arranged in a matrix and extend from the lower surface 50 of the base. In the configuration shown, the gripping structures are generally identical in configuration and are positioned in a matrix of three rows and eight columns extending on either side of the grasping opening 54. In the configuration shown, the gripping structures are separated from each other (both in columns and rows) by 0.100 inches. The leading edge of the gripping structures of the leading row in the direction of the leading edge of the base is spaced away from a central axis of the base (i.e., an axis bisecting the base of the mechanical coupling member) by a distance of 0.158 inches. Similarly, the leading edge of the gripping structures of the leading row in the direction of the trailing edge of the base is spaced away from a central axis of the base by a distance of 0.142 inches. Of course, other configurations are likewise contemplated. It is desirous to have a density of the gripping structures such that interference from adjacent gripping structures is minimized, while a sufficient number of gripping structures can be utilized to maximize retention and strength of the hold thereof.

The gripping structures 44 comprise a base configuration 60 and upstanding structure 70. The base configuration further comprises a leading edge 62, first rearward trailing edge 64, second rearward trailing edge 66, and rear intersection region 68. In the configuration shown, the base configuration 60 substantially defines a triangle, with the leading edge being substantially parallel to the leading edge 55 of the base 42, and generally perpendicular to the tip axis 188. The first and second rearward trailing edges 66, 68 together with the leading edge 55 define an isosceles triangle with the rear intersection region 68 defining the third point of the triangle. It has been found that a triangular cross-sectional configuration with the leading edge being perpendicular to the direction of force (i.e., the biasing force of the bandage or other structure to direct the wound to closure) and inboard of the rear intersection region exhibits improved strength characteristics and retention characteristics that a configuration that wherein the base configuration is flipped.

In the configuration shown, the leading edge is nominally 0.017 inches in width. The depth of the base structure, that is from the rear intersection region 68 to the leading edge is nominally 0.0156 inches. As such, width is slightly greater than the depth, defining a generally isosceles triangle.

The upstanding structure further comprises a front curved wall 71, first trailing wall 72, second railing wall 73, first upstanding edge 74, second upstanding edge 75, outer backbone 76, tip 78, and overhanging portion 80.

The front curved wall 71 extends from the leading edge 62 of the base. Similarly, the first trailing wall 72 extends from the first rearward trailing edge 64 and the second trailing wall 73 extends from the second rearward trailing edge 66. From these walls, the edges are created. That is, the first upstanding edge 74 is defined by the intersection between the front curved wall 71 and the first trailing wall 72. The second upstanding edge 75 is defined by the intersection between the front curved wall 71 and the second trailing wall 73. The outer backbone 76 is defined as the intersection between the first trailing wall 72 and second trailing wall 73 and extends from the rear intersection region 68. The intersection of all the walls extending from the base configuration defines the tip 78. The tip 78, due to the arcuate configuration of the outer back bone, and the first and second upstanding edges, defines a tip axis 88, which is generally controlled by the outer backbone the tip 78. In the configuration shown the tip axis is within a few degrees of being parallel to the lower surface of the base 42, and is preferably forms an angle α with a horizontal line intersecting at the tip of between −10° and 10°, and more preferably between −1° and 5°. It will be understood that in such a configuration, the axis is generally rather horizontal and facing in the direction of the force exerted by the inward biasing of the gripping structures.

In the configuration shown, the outer backbone is defined by multiple arcs, and in the configuration shown, three different arcuate configurations. The front curved wall is defined by multiple arcs, and in the configuration shown, two different arcuate configurations.

The overhanging portion 80 begins noticeably following the interface plane 82, or the plane perpendicular to the base 42 bisecting the upstanding structure 70 through the leading edge 62. Consequently, the base 83 of the interface plane 82 is colinear to the leading edge 62 of the base configuration 60. Following the interface plane 82 up the upstanding structure, the first edge 84 is the edge proximal to first side edge 24 and second edge 85 is the edge proximal to the second side edge 26. The intersection of these lines atop the upstanding structure 70 creates the top meeting region 86. In the configuration shown, the overall depth of the gripping structure, that is from the tip to the outer backbone at the base configuration is nominally 0.029 inches. The height of the tip from the base is nominally 0.0230 inches. When compared to the thickness of the base, which is nominally 0.005 inches, the height is 4.6 times the thickness of the base. Whereas the base has a thickness which is similar to the thickness (or slightly thicker or thinner) than the epidermis, the tip is intended to extend beyond the epidermis and into the dermis of the patient or user. As such, it is desirable that the height is preferably 2 times the thickness of the base, and more preferably 4 or more times the thickness of the base. It will be understood that in other configurations, the upstanding structure may be contained within the structure of the base configuration, and may not include an overhanging portion, while still including a tip as disclosed above that is directed toward the central region of the base substrate.

The overhang portion, that is the amount the tip is spaced apart from the leading edge is nominally 0.134 inches. Thus, the overhang portion extends beyond the footprint of the gripping structure (i.e., beyond the leading edge of the base configuration) by a distance that is approximately 85% of the depth of the gripping structure at the base configuration.

Attention should be given to the overhand portions 80 of each of the upstanding structures, as its complexity begets a difficulty in classic molding mechanisms when molded with the base 42. One skilled in the art may be familiar with the difficulty to mold materials with an overhand, or lip in the material, that can create a force to cement the molded object to the mold itself. The existence of these overhangs along the gripping structures, further added to the plurality of structures, creates a complex molding procedure. To mold the configuration shown, it is necessary to translate the mold in a direction that is perpendicular to the direction of removal of the member from the mold. That is, the molded member is first slid along the mold and then slid out of the mold. It may further be necessary to slightly rotate the molded member while being removed.

The adhesive coupling system 18, shown in FIG. 1, comprises first outbound adhesive portion 90, first inbound adhesive portion 92, second outbound adhesive portion 190, and second inbound adhesive portion 192. The first outbound adhesive portion 90 and first inbound adhesive portion 92 are generally within the first side region 36 and are positioned along opposite sides of the first side mechanical coupling member 40 such that the first outbound adhesive portion 90 is more proximal to the first end 20 than the leading edge 55 and the first inboard adhesive portion 92 is more proximal to the outer perimeter 29 of the wound covering member 14 than the trailing edge 56. The second outbound adhesive portion 190 and second inbound adhesive portion 192 are generally within the second side region 38 and are positioned along opposite sides of the second side mechanical coupling member 140 such that the second outbound adhesive portion 190 is more proximal to the second end 22 than the leading edge 155 and the second inboard adhesive portion 192 is more proximal to the outer perimeter 29 of the wound covering member 14 than the trailing edge 156.

The adhesive portions detailed in the adhesive coupling system 18 are substantially within the boundaries of the base substrate 12 such that the outer edges of the portions are preferably contained within the limits of the first side edge 24 and second side edge 26. The width and length dimensions of these adhesive portions are variable and dependent upon the sizing of the base substrate 12. Alterations to the base substrate 12 may or may not lead to size changes to the adhesive coupling system 18. Further, the adhesive used for the adhesive coupling system may be any skin friendly contact material. These adhesives are known to hypoallergenic and anti-inflammatory with respect to the skin surface to which the bandage 10 is likely to be applied. Those with skill in the art will appreciate the greater contact surface of adhesives such as those in the adhesive coupling system will lead to greater contact and mating with the skin surface.

It will further be understood that the adhesive coupling system may be omitted, in which case the bandage is secured to the user solely through the mechanical coupling system. In alternative designs, a tape or other material may be placed over the bandage to secure the bandage further to the user. In other configurations, either one of the inboard adhesive portion or the outboard adhesive portion may be omitted. In still further configurations a single adhesive coupling system 18 may be applied to the entirety (or substantially the entirety) of the inner surface 32 of the base substrate. In such a configuration, the adhesive can be utilized for coupling of the mechanical coupling system as well as the wound covering member.

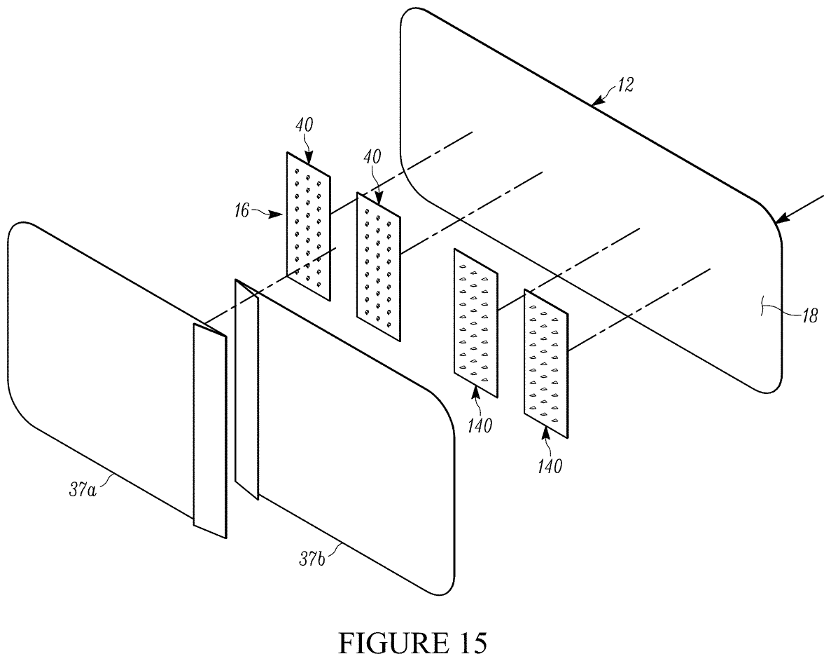

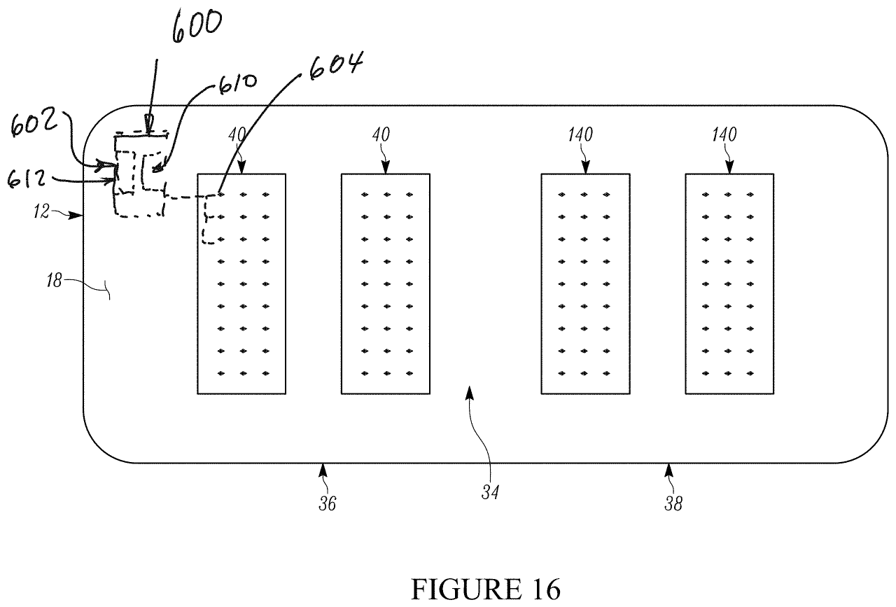

Another configuration of the bandage is shown in FIGS. 15 and 16. In the configuration shown therein, the wound covering member is omitted, and the mechanical coupling member includes a plurality of first side coupling members and a plurality of second side coupling members (which are sometimes referred to same side coupling members), in place of a single coupling member on both the first side and the second side. The coupling members on each side are spaced apart from each other. Additionally, in the configuration shown, the side edges of the base of each coupling member are spaced apart from the side edges of the base substrate a distance that is greater than in the configuration shown in FIG. 1. In addition, in FIG. 15, a protective, and removable cover 37a, 37b is disclosed that protects the adhesive prior to use of the bandage. Such removable covers are known to those of skill in the art. With sufficient base substrate, which is substantially entirely covered with adhesive (from the adhesive coupling system), the removable cover essentially encapsulates the mechanical coupling system between the cover and the underlying base substrate. Interestingly, the multiple mechanical coupling members on the first side may be biased relative to each other as they are separated from each other by a generally elastic base substrate. In other configurations, the base substrate may be of limited (or non-existent) elastic deformation, wherein the skin of the user can be utilized as a biasing force.

Detailed further is a proposed use for the bandage 10 and method of application. The method of application is illustrative solely, and other methods of application are contemplated, for example, depending upon these contemplated injuries. Injuries that the bandage is meant to address are superficial wounds damaging the skin. It is contemplated that multiple bandages 10 may be used in parallel to one another in order to increase the coverage of the contemplated wound. It is further contemplated that a number of particular types of bandages, including, for example, bandages which may wrap around a user, bandages which may include valving or the like, bandages which have a central wound covering member, with a surrounding mechanical and adhesive coupling system extending fully around the central wound covering member, as well as specialty bandage structures. It will be understood that variations are contemplated that incorporate the mechanical coupling system in a number of different bandage and base substrate configurations.

At the place of the wound, one of the mechanical coupling members of the mechanical coupling systems is coupled to the skin of the user (and, where provided, the adhesive coupling system is coupled to the skin). The mechanical coupling member is introduced at a location on one side of a wound such that when coupled to the skin, the bandage extends across the wound, with the wound covering member extending over the wound itself.

Next, the user can grasp the non attached end of the bandage and pull the bandage, generally aiding in closing the wound, and also, in some instances, stretching, elastically, the base substrate. To better grasp the bandage, the user can place a finger through the grasping opening and pulling the same. Once stretched, the user can direct the second side mechanical coupling member toward the skin so that the gripping structures pierce the skin. The bias created by the elastic nature of the base substrate, or simply the compressing of the skin prior to the application of the second side mechanical coupling member applies an inwardly directed force. Movement, or that force, is countered by the gripping structures and their interaction with the dermis and epidermis of the user.

Finally, the adhesive coupling system 18 can maintain the bandage in the desired orientation. In other configurations, other bandages, tape, wraps or the like may be provided over the bandage. In still other configurations, additional wound coverings may be employed.

In some configurations of the bandage shown and described (including wound care devices in the incorporated references above), may be augmented with a system 600 for monitoring the wound. With reference to FIG. 16, the system 600 includes communication device 602, and at least one sensor, such as sensor 604. The communication device 602 can be mounted on a number of different locations on the bandage, such as on the outside surface of the substrate 12. The communication device 602 may comprise a display member 610 and/or a transmitting device 612. The display member can provide the status of the sensors so as to provide an indication of the condition of the region around the wound. In some configurations, the display member may comprise a color change to a display member, or a circuit that is completed to provide an audible, visual or tactile signal.

The transmitting device 612 may comprise circuitry that provides communication to outside devices. Such a transmitting device may comprise circuitry that can send communication (and receive communication) in the form of, for example, Bluetooth, wifi, zigbee, and other short or longer range communication with a device (such as a computer, tablet and/or smartphone.

With reference to FIG. 12, the sensor, such as one of sensors 604a-d, may extend into one or more gripping structures 44. For example, a bore may extend through the gripping structure, or a channel or the like along a gripping structure wherein a sensor can be applied or provided. Such a sensor may comprise a chemical or electrical sensor, among others. The sensor may sense such conditions as temperature, humidity, water content, the presence or absence of certain chemicals, compounds or other elements. The disclosure is not limited to any particular type of sensor. It will be understood that in some applications, the sensor may be positioned so as to be in contact with the patient and within the bore of the gripping structure. In other configurations, the sensor may be positioned so as to be in communication with the bore, and the bore is utilized as a point of collection or sending environment for the sensor. In still other configurations, the sensor may be associated with the surface 40 from which the gripping structures emanate.

In the embodiment of FIG. 12, four different sensors at different locations are shown. For example, a sensor 604a is positioned at the tip 78 of the gripping structure. Sensor 604b is positioned within the bore formed within the gripping structure 44. Sensor 604c is positioned at or near the base of the gripping structure 44. Sensor 604d is positioned on the base of the mechanical coupling member. Of course, these are exemplary and other locations are contemplated. Moreover, while four sensors are shown, it will be understood that in many cases a gripping structure may have or be associated with only one sensor. That is, a single sensor may be present on the entire mechanical coupling member, or multiple sensors may be associated with the mechanical coupling member, but these may be on different gripping structures or on the base. The particular location and number of sensors that are utilized can be varied based on embodiment and position (i.e., subcutaneous versus on the skin, etc.), and size of the device.

It will be understood that once the bandage is applied (or the wound closure is applied subcutaneously), the sensor is activated and operational. For example, where the sensor is a temperature sensor, the system may be configured to trigger the display member, or to send a communication if the temperature that is sensed is outside of a particular set range. In some configurations, this may cause a portion of the display member 610 to change color. In other configurations, a signal may be sent from the system to a smartphone, a tablet or a computer (among other computing devices) as an alert or the like. Analogous descriptions may be provided for a number of different types of sensors. That is, the system will trigger an indication in the form of a change in the display member, or a signal that is transmitted, if the sensed condition is in a condition that has been identified or a condition wherein some type of alert or notification is desired.

The foregoing description merely explains and illustrates the disclosure and the disclosure is not limited thereto except insofar as the appended claims are so limited, as those skilled in the art who have the disclosure before them will be able to make modifications without departing from the scope of the disclosure.

What is claimed is:

1. A wound closure device comprising:
a mechanical coupling system comprising a plurality of gripping structures; and
a system for monitoring a wound including a communication device and a sensor.
